(19) United States
(12) Patent Application Publication
(10) Pub. No.: US 2008/0234302 A1
(43) Pub. Date: Sep. 25, 2008

(54) Novel Processes for Preparing Amorphous Rosuvastatin Calcium and a Novel Polymorphic Form of Rosuvastatin Sodium

(76) Inventors: Mohammad Rafeeq, Uttar Pradesh (IN); Shantanu De, New Delhi (IN); Swargam Sathyanarayana, Andhra Pradesh (IN); Yatendra Kumar, Haryana (IN)

Correspondence Address: RANBAXY INC., 600 COLLEGE ROAD EAST, SUITE 2100, PRINCETON, NJ, 08540, US

(21) Appl. No.: 11/575,817

(22) PCT Filed: Sep. 20, 2005
(86) PCT No.: PCT/IB2005/002784

§ 371 Date: Mar. 17, 2008

(30) Foreign Application Priority Data
Sep. 27, 2004 (IN) 1844/DEL/2004
Sep. 27, 2004 (IN) 1845/DEL/2004

(51) Int. Cl. A61K 31/505; C07D 239/42; A61P 3/06

(52) U.S. Cl. 514/275; 544/332

(57) ABSTRACT

Provided are processes for preparing amorphous rosuvastatin calcium from crystalline rosuvastatin calcium by simple precipitation processes. Also provided is a novel polymorphic form of rosuvastatin sodium, processes for preparing thereof and pharmaceutical compositions thereof.

### FIELD OF THE INVENTION

Provided are processes for preparing amorphous rosuvastatin calcium from crystalline rosuvastatin calcium by simple crystallization processes. Also provided is a novel polymorphic form of rosuvastatin sodium, processes for preparing thereof and pharmaceutical compositions thereof.

### BACKGROUND OF THE INVENTION

Rosuvastatin calcium is chemically, (3R,5S,6E)-7-[4-(4-fluorophenyl)-6-(1-methylethyl)-2-[methyl(methylsulfonyl)amino]-5-pyrimidinyl] -3,5-dihydroxy-6-heptenoic acid, calcium salt of Formula Ia and rosuvastatin sodium is chemically, (3R,5S,6E)-7-[4-(4-fluorophenyl)-6-(1-methylethyl)-2-[methyl(methylsulfonyl)amino]-5-pyrimidinyl]-3,5-dihydroxy-6-heptenoic acid, sodium salt of Formula Ib.

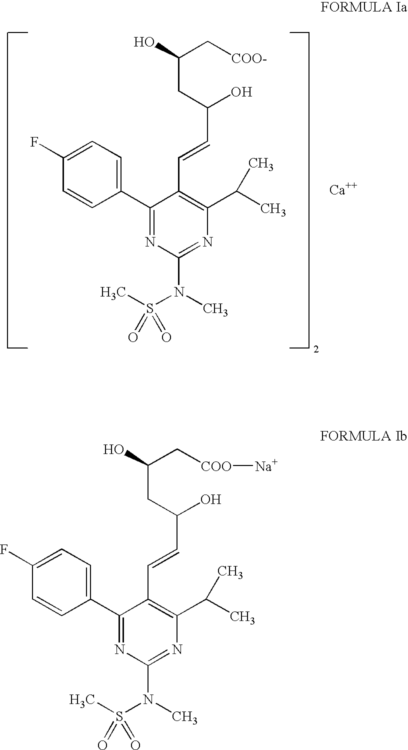

Rosuvastatin and its salts are inhibitors of HMG-CoA enzyme. Rosuvastatin calcium of Formula Ia and Rosuvastatin sodium of Formula Ib are antihypercholesterolemic drugs used in the treatment of atherosclerosis. Hypercholesterolemia is now well recognized as a primary risk in coronary heart disease. Clinical studies with lipid lowering agents have established that decreasing elevated serum cholesterol level reduces the incidence of cardiovascular mortality. First generation drugs for the treatment of atherosclerosis by inhibiting the activity of HMG-CoA reductase include, for example, pravastatin and simvastatin, which are fungal metabolites or chemical modifications thereof. Recently developed synthetic inhibitors of HMG-CoA reductase include, for example, fluvastatin, and are considered as second generation drugs.

U.S. Pat. No. RE37,314 discloses a process for preparing amorphous rosuvastatin calcium by converting rosuvastatin lactone or rosuvastatin ester to its sodium salt by treatment with sodium hydroxide in water to form rosuvastatin sodium followed by adding calcium chloride and collecting the resultant precipitate by filtration.

U.S. Pat. No. 6,589,959 discloses a process for preparing crystalline form A of rosuvastatin by warming the amorphous form of rosuvastatin calcium in a mixture of water and acetonitrile, cooling the resultant solution to ambient temperature and then filtering the product which is then dried at 50° C. under vacuum to give crystalline Form A of rosuvastatin calcium.

PCT Publication WO 2005/040134 provides several cost-effective and simple methods for preparing amorphous form of rosuvastatin calcium.

Other publications disclose preparing rosuvastatin calcium by treating in situ formed rosuvastatin sodium with calcium ions. However, there remains a need for other processes to prepare rosuvastatin calcium.

### BRIEF DESCRIPTION OF THE FIGURES

FIG. 1 is an X-ray powder diffraction (XRD) pattern of amorphous rosuvastatin calcium.

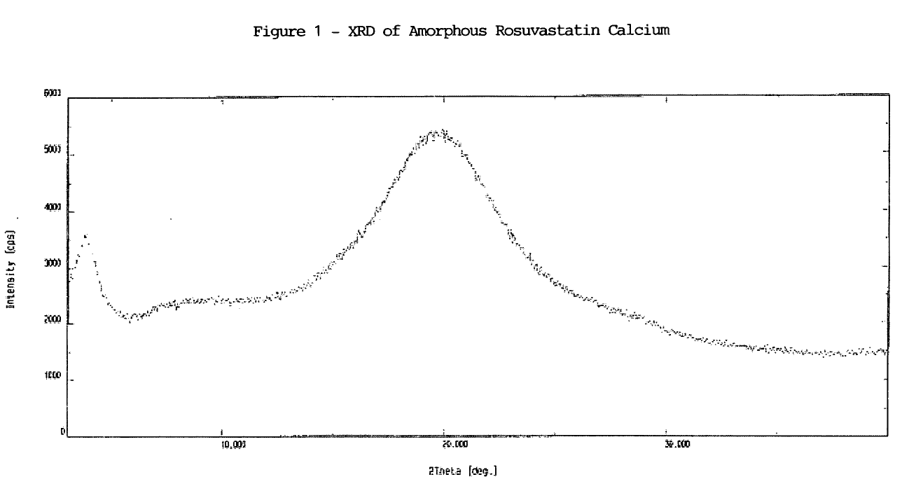

FIG. 2 is an X-ray powder diffraction (XRD) pattern of crystalline rosuvastatin calcium.

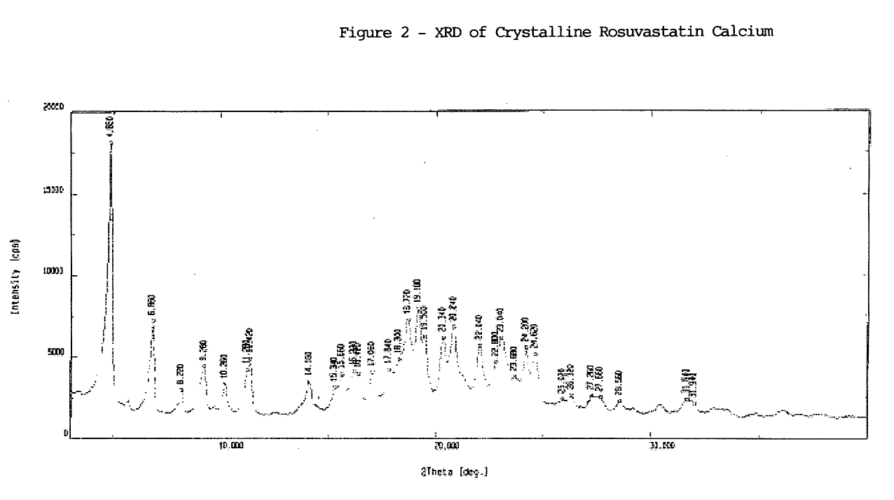

FIG. 3 is an X-ray powder diffraction (XRD) pattern of Form A of rosuvastatin sodium.

### SUMMARY OF THE INVENTION

It has been surprisingly discovered that substantially pure amorphous rosuvastatin calcium can be prepared by simple crystallization methods. The methods described herein offer advantages including cost, equipment type and configurations and scalability in preparing amorphous rosuvastatin calcium. It has also been found that rosuvastatin sodium can be isolated in novel crystalline form having significantly high purity, which provides highly pure rosuvastatin calcium when treated with calcium ions. The novel polymorphic form of rosuvastatin sodium is highly stable and can be used as a HMG-CoA enzyme inhibitor.

Thus in one aspect, provided are processes for preparing amorphous rosuvastatin calcium comprising the steps of:
a) dissolving crystalline rosuvastatin calcium in one or more organic solvents to form a mixture,
b) flash cooling the mixture to about 10 to −50° C. to form amorphous rosuvastatin calcium, and
c) isolating amorphous rosuvastatin calcium from mixture thereof.

The processes can include one or more of the following embodiments. For example, the one or more organic solvents can be selected from one or more lower alcohols, for example, methanol, ethanol, n-propanol, isopropanol, n-butanol, isobutanol, tert-butanol or mixtures thereof.

In another aspect, provided are processes for preparing amorphous rosuvastatin calcium comprising the steps of:
a) dissolving crystalline rosuvastatin calcium in one or more organic solvents and optionally water to form a mixture,
b) removing about 40 to 85% v/v of the one or more organic solvents and optionally water from the mixture thereof to form a concentrated mixture,
c) cooling the concentrated mixture to about 0 to 30° C.,
d) isolating amorphous rosuvastatin calcium from the concentrated mixture.

The processes can include one or more of the following embodiments. For example, the one or more organic solvents can be selected from one or more lower alcohols, for example, methanol, ethanol, n-propanol, isopropanol, n-butanol, isobutanol, tert-butanol or mixtures thereof.

In another aspect, provided are processes for preparing amorphous rosuvastatin calcium comprising the steps of:
a) dissolving rosuvastatin calcium in isopropanol to form a mixture,
b) heating the mixture to about a temperature from about 50° C. to reflux temperature,
c) slowly cooling the mixture to ambient temperature and isolating amorphous rosuvastatin calcium from mixture thereof.

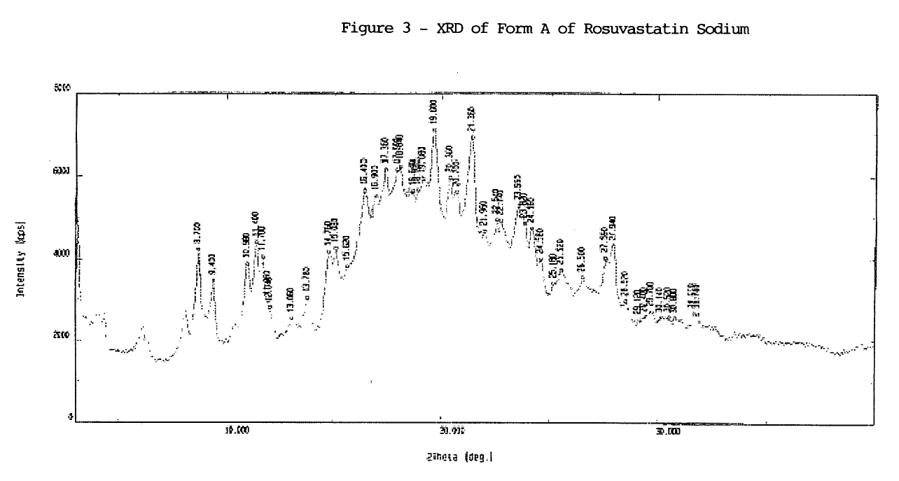

In another aspect, provided is Polymorphic Form A of rosuvastatin sodium. Form A of rosuvastatin sodium can include one or more of the following embodiments. For example, Form A of rosuvastatin sodium of claim 7 can exhibit an X-Ray Diffraction (XRD) pattern having one or more 2θ values at about 8.7, 11.4, 19.6 and 21.4. Form A of rosuvastatin sodium can also exhibit an X-Ray Diffraction (XRD) pattern having one or more 2θ values of about 9.4, 11.0, 14.8, 15.1, 16.4, 17.4, 23.6 and 27.9. Form A of rosuvastatin sodium can also exhibit an X-Ray Diffraction (XRD) pattern having one or more 2θ values at about 11.7, 12.0, 12.0, 13.1, 13.8, 15.6, 16.9, 17.4, 17.9, 18.0, 18.6, 18.9, 19.1, 20.4, 20.7, 22.0, 22.5, 22.7, 23.8, 24.2, 24.6, 25.2, 25.5, 26.5, 27.6, 28.5, 29.1, 29.4, 29.7, 30.1, 30.5, 30.8, 31.6 and 31.8. For example, Form A of rosuvastatin sodium can exhibit an X-Ray Diffraction (XRD) pattern as depicted in FIG. 3.

In another aspect, provided is substantially pure rosuvastatin sodium having purity above 98% by HPLC.

In another aspect, also provided are processes for preparing polymorphic Form A of rosuvastatin sodium comprising the steps of:
a) contacting rosuvastatin methyl ammonium salt of Formula II with one or more acids to form rosuvastatin acid of Formula III;

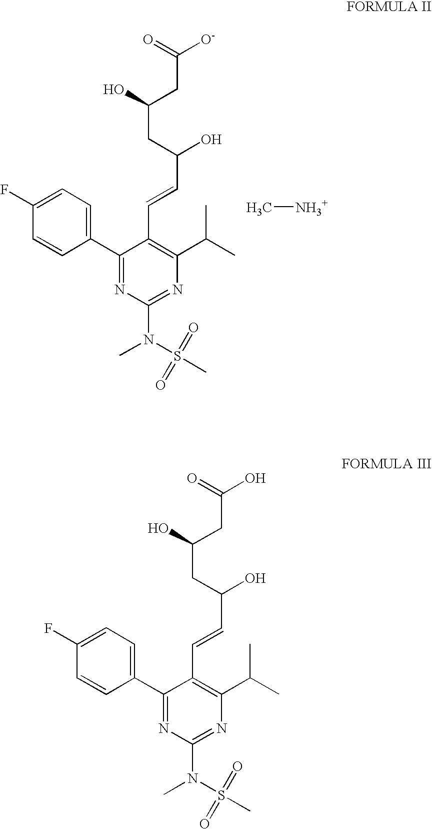

b) contacting rosuvastatin acid of Formula III with one or more sodium-containing bases to form rosuvastatin sodium, and
c) adding one or more antisolvents and a catalytic amount of water to rosuvastatin sodium of step b) and recovering crystalline rosuvastatin sodium.

The process can include one or more of the following embodiments. For example, the one or more sodium-containing bases can be selected from one or more of sodium hydroxide, sodium carbonate, sodium bicarbonate or mixtures thereof. The one or more antisolvents can be selected from one or more of diethyl ether, methyl tert-butyl ether, diisopropyl ether, hexane, heptane, cyclohexane, cycloheptane, petroleum ether or mixtures thereof.

In another aspect, also provided are processes for preparing rosuvastatin calcium or rosuvastatin magnesium comprising the steps of:
a) contacting Form A of rosuvastatin sodium with calcium ions or magnesium ions in presence of water and optionally one or more organic solvents to form a mixture, and
b) isolating rosuvastatin calcium or rosuvastatin magnesium from the mixture thereof.

The processes can include one or more of the following embodiments. For example, the calcium ions in step a) can be provided by one or more calcium-containing compounds and the magnesium ions used in step a) can be provided by one or more magnesium-containing compounds. Suitable calcium-containing compounds include, for example, calcium chloride, calcium hydroxide, calcium acetate or mixtures thereof. Suitable magnesium-containing compounds include, for example, magnesium hydroxide, magnesium sulphate, magnesium acetate, magnesium chloride, magnesium oxide, magnesium lactate or mixtures thereof.

In another aspect, provided are pharmaceutical compositions comprising Form A of rosuvastatin sodium and optionally one or more pharmaceutically acceptable excipients or diluents.

In another aspect, provided are methods of antagonizing HMG-CoA enzyme, which comprises administering to a mammal in need thereof a therapeutically effective amount of Form A of rosuvastatin sodium.

### DETAILED DESCRIPTION OF THE INVENTION

One aspect provides processes for preparing amorphous rosuvastatin calcium comprising the steps of:
a) dissolving crystalline rosuvastatin calcium in one or more organic solvents to form a mixture,
b) flash cooling the mixture to about 10 to −50° C. to form amorphous rosuvastatin calcium, and
c) isolating amorphous rosuvastatin calcium from the mixture thereof.

Suitable solvents for dissolving crystalline rosuvastatin calcium can include lower alcohols, i.e., C₁-C₆ alcohols, for example, selected from one or more of methanol, ethanol, n-propanol, isopropanol, n-butanol, isobutanol, tert-butanol or mixtures thereof. A preferred lower alcohol is ethanol. The solution can be flash cooled to quickly decrease the temperature of the solution. Preferably the solution can be cooled to about 5 to −20° C., more preferably to about 0° C.

Another aspect provides processes for preparing amorphous rosuvastatin calcium comprising the steps of:
a) dissolving crystalline rosuvastatin calcium in one or more organic solvents to form a mixture,
b) removing about 40 to 85% v/v of the one or more organic solvents from the mixture thereof to form a concentrated mixture,
c) quickly cooling the concentrated mixture to about 0 to 30° C., and
d) isolating amorphous rosuvastatin calcium from the concentrated mixture after stirring.

Suitable organic solvents to dissolve crystalline rosuvastatin include one or more lower alcohols and optionally water. Lower alcohols can include C₁-C₆ alcohols, for example, methanol, ethanol, n-propanol, isopropanol, n-butanol, isobutanol, tert-butanol or mixtures thereof. After crystalline rosuvastatin is contacted with the one or more organic solvents, the mixture can be heated up to reflux temperature. Water can be added to the mixture to facilitate dissolution. About 45 to about 85% of the solvent can be removed from the mixture by distillation to form a concentrated mixture. The resultant concentrated mixture can be cooled to ambient temperatures and stirred for time sufficient to form amorphous rosuvastatin calcium. Stirring times can range from about 1 to 48 hours, from about 4 to 24 hours, from about 6 to 12 hours and even about 8 hours.

Another aspect provides processes for preparing amorphous rosuvastatin calcium comprising the steps of:
a) dissolving rosuvastatin calcium in isopropanol to form a mixture,
b) heating the mixture to a temperature from about 50° C. to reflux temperature,
c) slowly cooling the mixture to ambient temperature and isolating amorphous rosuvastatin calcium from the mixture thereof.

Crystalline rosuvastatin calcium can be dissolved in isopropanol at reflux temperature and the resultant mixture can be slowly cooled to ambient temperature with stirring. Amorphous rosuvastatin calcium can be isolated from mixture by filtration and dried by conventional means.

Also provided is novel polymorphic Form A of rosuvastatin sodium. Form A of rosuvastatin sodium can exhibit an X-Ray Diffraction (XRD) pattern having one or more 2θ values at about: 8.7, 11.4, 19.6, and/or 21.4. Form A of rosuvastatin sodium can also exhibit an XRD pattern having one or more 2θ values at about: 9.4, 11.0, 14.8, 15.1, 16.4, 17.4, 23.6 and/or 27.9. Form A of rosuvastatin sodium can also exhibit an XRD pattern having one or more 2θ values at about: 11.7, 12.0, 12.0, 13.1, 13.8, 15.6, 16.9, 17.4, 17.9, 18.0, 18.6, 18.9, 19.1, 20.4, 20.7, 22.0, 22.5, 22.7, 23.8, 24.2, 24.6, 25.2, 25.5, 26.5, 27.6, 28.5, 29.1, 29.4, 29.7, 30.1, 30.5, 30.8, 31.6 and 31.8.

Another aspect provides substantially pure rosuvastatin sodium having purity above 98% when measured by HPLC. In other embodiments, substantially pure rosuvastatin sodium can have purity above about 96%, above about 97%, above about 98% and even above about 99%.

Another aspect provides processes for preparing novel polymorphic Form A of rosuvastatin sodium comprising the steps of:
a) contacting rosuvastatin methyl ammonium salt of Formula II with one or more acids to form rosuvastatin acid of Formula III;

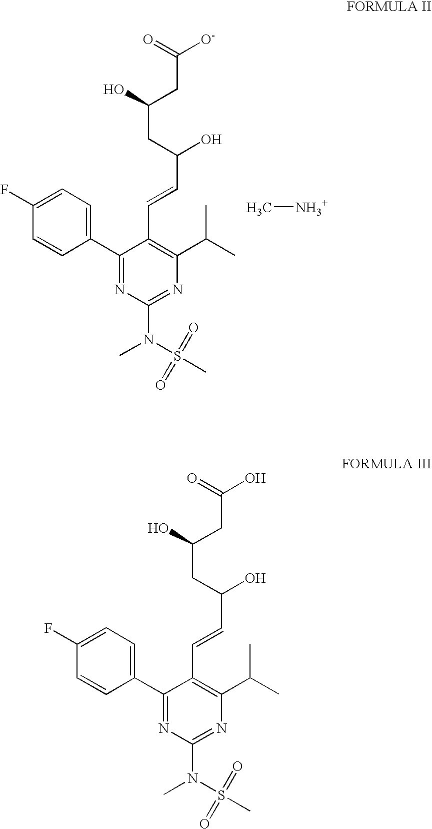

b) contacting rosuvastatin acid of Formula III with one or more sodium-containing bases to form rosuvastatin sodium, and
c) adding one or more antisolvents and a catalytic amount of water to rosuvastatin sodium of step b) and recovering crystalline rosuvastatin sodium.

Rosuvastatin methyl ammonium salt of Formula II can be prepared by, for example, the process disclosed in PCT application WO 01/60804. Rosuvastatin methyl ammonium salt of Formula II can then be contacted with one or more acids in one or more first organic solvents and optionally water to form a first mixture and the first mixture is brought to temperatures between about −10 to 100° C. The one or more acids can be added to lower the pH of the reaction to about 1 to 5. After completion of reaction, the organic layer can be separated and washed with water and/or brine, and the solvent can be removed completely under vacuum. Suitable acids can include, for example, one or more inorganic mineral acids (for example hydrochloric acid, sulfuric acid, nitric acid, phosphoric acid or mixtures thereof), one or more organic acids (for example, formic acid, acetic acid and the like or mixtures thereof) or mixtures thereof. Suitable first organic solvents can include, for example, one or more water immiscible and/or partially miscible organic solvents (for example, toluene, xylene, benzene, ethyl methyl ketone, diisobutyl ketone, methyl isobutyl ketone, methyl tert-butyl ether, diisopropyl ether, ethyl acetate, methyl formate, methyl acetate, isobutyl acetate, n-propyl acetate, isopropyl acetate, amyl acetate or mixtures thereof).

Rosuvastatin acid of Formula III can be dissolved in one or more second organic solvents and optionally water and contacted with one or more sodium-containing bases to form a second mixture. The second mixture can be brought to temperatures of about 10 to 70° C. for about 1 to 40 hours to facilitate hydrolysis of the lactone. The pH of the second mixture can be about 7.5 to 11. Sodium-containing bases include, for example, one or more of sodium hydroxide, sodium carbonate, sodium bicarbonate or mixtures thereof.

Suitable second organic solvents include, for example, one or more lower alcohols, one or more polar aprotic solvents (for example, C₃-C₁₀ ketones, C₃-C₆ ethers, nitriles) or mixtures thereof.

Solvent can be removed from the second mixture to leave a concentrated mass. The concentrated mass can be contacted with one or more antisolvents containing catalytic amounts of water to yield novel polymorphic Form A of rosuvastatin sodium. Antisolvents include solvents in which rosuvastatin sodium is insoluble, practically insoluble or sparingly insoluble. Suitable antisolvents include, for example, one or more of diethyl ether, methyl tert-butyl ether, diisopropyl ether, hexane, heptane, cyclohexane, cycloheptane, petroleum ether or mixtures thereof. The product thus obtained can be dried by conventional means including, for example, under vacuum at ambient temperature.

Also provided are processes for preparing rosuvastatin calcium or rosuvastatin magnesium comprising the steps of:
a) contacting Form A of rosuvastatin sodium with calcium ions or magnesium ions in presence of water and optionally one or more organic solvents to form a mixture, and
b) isolating rosuvastatin calcium or rosuvastatin magnesium from the mixture thereof.

Rosuvastatin sodium Form A can be converted to rosuvastatin calcium or rosuvastatin magnesium by contacting rosuvastatin sodium Form A with one or more suitable calcium or magnesium-containing compound in aqueous conditions. Suitable calcium-containing compounds include, for example, calcium chloride, calcium hydroxide, calcium acetate or mixtures thereof. Suitable magnesium-containing compounds include, for example, one or ore magnesium hydroxide, magnesium sulphate, magnesium acetate, magnesium chloride, magnesium oxide, magnesium lactate or mixtures thereof.

Also provided are pharmaceutical compositions comprising Form A of rosuvastatin sodium and optionally one or more pharmaceutically acceptable excipients or diluents.

Also provided are methods of antagonizing HMG-CoA enzyme, which comprises administering to a mammal in need thereof therapeutically effective amounts of Form A of rosuvastatin sodium.

While the present invention has been described in terms of its specific embodiments, certain modifications and equivalents will be apparent to those skilled in the art and are included within the scope of the present invention. The examples are provided to illustrate particular aspects of the disclosure and do not limit the scope of the present invention as defined by the claims.

### EXAMPLES

### Example 1

### Preparation of Amorphous Rosuvastatin Calcium

Crystalline rosuvastatin calcium (20 g) was added to denatured spirit (40 mL) and the resultant mixture was stirred for 10 minutes at ambient temperature and then heated to about 77° C. to form produce a clear solution. The clear solution was immediately cooled to about 0° C. over 10 minutes. The resultant suspension was stirred at 0° C. for 30 minutes. The separated product was filtered and dried under vacuum at about 40-45° C. to yield amorphous rosuvastatin calcium.

### Yield: 1.3 g (65%)

### HPLC Purity: 99.72%

### Example 2

### Preparation of Amorphous Rosuvastatin Calcium

Crystalline rosuvastatin calcium (5.0 g) was dissolved in isopropanol (300 mL) at about 25-30° C. The mixture was heated to reflux and then de-ionized water (1 mL) was to form a clear solution. Isopropanol (about 250 mL) was removed from the mixture under atmospheric pressure with heating at about 80-85° C. and the resultant mass was slowly cooled to ambient temperature over 1 hour, forming a sticky material. The mixture was then stirred for about 8 hours at ambient temperature and then cooled to about 3-4° C. The product thus obtained was filtered and washed with isopropanol (10 mL) and dried under vacuum at 45° C. to yield amorphous rosuvastatin calcium.

### Yield: 3.9 g (79%)

### Example 3

### Preparation of Amorphous Rosuvastatin

Crystalline rosuvastatin calcium (2.0 g) was dissolved in isopropanol at reflux temperatures and the resultant mixture was slowly cooled to ambient temperature over 1 hour, forming a sticky material. The mixture was further stirred at ambient temperature for 8 hours and filtered, washed with isopropanol and dried under vacuum at 40-45° C. to yield amorphous rosuvastatin calcium.

### Yield: 1.2 g (60%)

### Example 4

### Preparation of Form A of Rosuvastatin Sodium

Step A) Preparation of Rosuvastatin Acid from Rosuvastatin Methyl Ammonium Salt.

Rosuvastatin methyl ammonium salt (8 g) was added to ethyl acetate (50 mL) and de-ionized water (40 mL) at 25-30° C. and the pH was adjusted to about 4.0 with 6N hydrochloric acid. The aqueous and organic layers thus formed were separated and the organic layer was washed with deionized water (50 mL). The organic layer was then concentrated by complete removal of solvent under vacuum to yield the title compound as an oil.

### Step B) Conversion of Rosuvastatin Acid to Crystalline Rosuvastatin Sodium.

Rosuvastatin acid as obtained in step A) was dissolved in methanol (40 mL) and water (50 mL) to form a mixture. A sodium hydroxide solution (8% w/v) was added to the mixture until the pH was about 9.0 and the basified mixture stirred for 30 minutes. The solvent was removed under vacuum and the crude product thus obtained was triturated with n-hexane (50 mL). The n-hexane was decanted, the product thus obtained was subjected to high vacuum for 1 hour, and diethyl ether (50 mL) and 2 drops of water were added. The resultant mixture was stirred for 12 to 14 hours at ambient temperature. The separated solids were filtered and dried under vacuum at ambient temperature to yield crystalline Form A of rosuvastatin sodium.

Yield: 6.0 g (XRD as per FIG. 3).

We claim:

1. A process for preparing amorphous rosuvastatin calcium comprising the steps of:
a) dissolving crystalline rosuvastatin calcium in one or more organic solvents to form a mixture,
b) flash cooling the mixture to about 10 to −50° C. to form amorphous rosuvastatin calcium, and
c) isolating amorphous rosuvastatin calcium from mixture thereof.

2. The process of claim 1, wherein the one or more organic solvents are selected from one or more lower alcohols.

3. The process of claim 2, wherein the one or more lower alcohols are selected from methanol, ethanol, n-propanol, isopropanol, n-butanol isobutanol, tert-butanol or mixtures thereof.

4. A process for preparing amorphous rosuvastatin calcium comprising the steps of:
a) dissolving crystalline rosuvastatin calcium in one or more organic solvents and optionally water to form a mixture,
b) removing about 40 to 85% v/v of the one or more organic solvents and optionally water from the mixture thereof to form a concentrated mixture,
c) cooling the concentrated mixture to about 0 to 30° C.,
d) isolating amorphous rosuvastatin calcium from the concentrated mixture

5. The process of claims 4, wherein the one or more organic solvents are selected from one or more lower alcohols.

6. The process of claim 5, wherein the one or more lower alcohols are selected from methanol, ethanol, n-propanol, isopropanol, n-butanol, isobutanol, tert-butanol or mixtures thereof.

7. A process for preparing amorphous rosuvastatin calcium comprising the steps of:
a) dissolving rosuvastatin calcium in isopropanol to form a mixture,
b) heating the mixture to about a temperature from about 50° C. to reflux temperature,
c) slowly cooling the mixture to ambient temperature and isolating amorphous rosuvastatin calcium from mixture thereof.

8. Polymorphic Form A of rosuvastatin sodium.

9. Form A of rosuvastatin sodium of claim 8 exhibiting an X-Ray Diffraction (XRD) pattern having one or more 2θ values at about 8.7, 1.14, 19.6 and 21.4.

10. Form A of rosuvastatin sodium of claim 8 exhibiting an X-Ray Diffraction (XRD) pattern having one or more 2θ values of about 9.4, 11.0, 14.8, 15.1, 16.4, 17.4, 23.6 and 27.9.

11. Form A of rosuvastatin sodium of claim 8 exhibiting an X-Ray Diffraction (XRD) pattern having one or more 2θ values at about 11.7, 2.0, 12.0, 13.1, 13.8, 15.6, 16.69, 17.4, 17.99, 18.0, 18.6, 18.9, 19.1, 20.4, 20.7, 22.0, 22.5, 22.7, 23.8, 24.2, 24.6, 25.2, 2.55, 26.5, 27.96, 28.5, 29.1, 29.4, 29.7, 30.1, 30.5, 30.8, 31.6 and 31.8.

12. Form A of rosuvastatin sodium of claim 8 exhibiting an X-Ray Diffraction (XRD) pattern as depicted in FIG. 3.

13. Substantially pure rosuvastatin sodium having purity above 98% by HPLC.

14. A process for preparing polymorphic Form A of rosuvastatin sodium comprising the steps of:
a) contacting rosuvastatin methyl ammonium salt of Formula II with one or more acids to form rosuvastatin acid of Formula III; 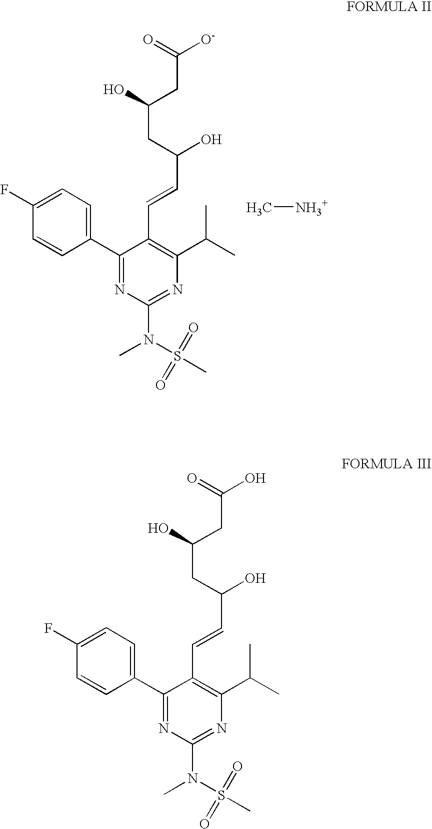
b) contacting rosuvastatin acid of Formula III with one or more sodium-containing bases to form rosuvastatin sodium, and
c) adding one or more antisolvents and a catalytic amount of water to rosuvastatin sodium of step b) and recovering crystalline rosuvastatin sodium.

15. The process of claim 14, wherein the one or more sodium-containing bases are selected from one or more of sodium hydroxide, sodium carbonate, sodium bicarbonate or mixtures thereof.

16. The process of claim 15, wherein the one or more antisolvents are selected from one or more of diethyl ether, methyl tert-butyl ether, diisopropyl ether, hexane, heptane, cyclohexane, cycloheptane, petroleum ether or mixtures thereof.

17. (canceled)

18. (canceled)

19. (canceled)

20. (canceled)

21. A pharmaceutical composition comprising Form A of rosuvastatin sodium and optionally one or more pharmaceutically acceptable excipients or diluents.

22. A method of antagonizing HMG-CoA enzyme, which comprises administering to a mammal in need thereof a therapeutically effective amount of Form A of rosuvastatin sodium.
